(19) United States
(12) Patent Application Publication
(10) Pub. No.: US 2007/0066571 A1
(43) Pub. Date: Mar. 22, 2007

(54) Novel bisphosphonates having anti-inflammatory and/or pain-killing properties

(76) Inventors: Richard F. Stockel, Bridgewater, NJ (US); Edward G. Budnick, Flemington, NJ (US)

Correspondence Address: RICHARD F. STOCKEL, 475 ROLLING HILLS ROAD, BRIDGEWATER, NJ, 08807, US

(21) Appl. No.: 11/228,870

(22) Filed: Sep. 17, 2005

(51) Int. Cl. A61K 31/675; C07F 9/6506; C07F 9/58

(52) U.S. Cl. 514/89; 514/102; 514/95; 546/22; 548/112

(57) ABSTRACT

This invention describes new and novel bisphosphonate conjugates having anti-inflammatory and/or pain-killing properties.

Bisphosphonates (formerly known as diphosphonates) are analogues of pyrophosphates in which the oxygen atom of the central P-O-P structure has been replaced by carbon, resulting in a P-C-P group. This structure has made them resistant to heat and enzymatic hydrolysis and these phosphonates exert a strong effect on the bones.

Many bisphosphonates have been evaluated in animals and humans with respect to their effect on bone resorption (alendrimate, chlorinate, ethidronate, ibandronate, pamidronate, risedronate, and ibandronate). We have chemically combined analgesics and/or pain-killing narcotics, with the bisphosphonate, heretofore called the agents. This is exemplified by the following chemical reactions scheme. The following bisphosphonates, alendronate, EB-1053 (Leo), 1-Hydroxyl-2-(1-pyrrolidinyl)-propylidene) bis-phosphonate, etidronate, ibandronate, minodronate, opadronate, risedronate, zoledronate, all have in common a hydroxyl group on the carbon methylene bridge between the two phosphonic or phosphonate groups. Many analgesic materials can be used, the only provision being that they possess a carboxylic acid or ester group or a salt of the acid or an acid chloride. Salicylic acid, acetysalicylsalicylic acid and many other materials can be used. If salicylic acid is used it can be acylated with an appropriate acid anhydride; examples are acetic anhydride, propionic anhydride, butyric anhydride, valeric anhydride, pivalyl anhydride.

The following bisphosphonates, alendronate, incadronate, neridronate, pamidronate, all have one or more available hydrogen atoms bound to a nitrogen group. The amino group on the aforementioned bisphosphonates can be reacted with the acid chloride of aspirin or ibuprofen or other analgesic agents, resulting in a bisphosphonate, which has the analgesic agent chemically bound to the bisphosphonate.

The following bisphosphonates EB-1053, ibandronate, olpadronate, risedronate, zoledronate, can be reacted with the free acid form of an analgesic agent to give a quaternary ammonium salt of the analgesic agent. The term analgesic includes narcotics having an amine (one or more) in its structure like codeine, lorfan, talivin the like which can give quaternary salts with the phosphonic acid functionality. Clodronate has two chlorine atoms on the methylene bridge, which can enter into reactions to yield a variety of different analgesic bisphosphonates, for example a chloro group can replaced by a sodium carboxylate of a analgesic or narcotic agent. To achieve improved yields the chloro group can be replaced by a neucleophide displacement with a tosylate, followed by a second nucleophilic reaction with a sodium salt of a carboxylate.

Icandronate has one available hydrogen atom on the methylene bridge which can be replaced by a halogen atom such as chlorine bromine or iodine which is then reacted with the free acid form or the sodium or potassium salt of the free acid to yield a variety of analgesic bisphosphonates. The same available hydrogen can be replaced by a sodium or potassium metal and then reacted with the acid chloride form of the analgesic agent to yield a variety of analgesic bisphosphonates.

Tiludronate has an available hydrogen on the methylene bridge which can be replaced with a halogen reacted with the free acid or the sodium or potassium salt of the free acid of aspirin, ibuprofen or analgesic derivates possessing carboxylic group. Alternatively the available hydrogen can be replaced by sodium or potassium metal and then reacted with the acid chloride form of the analgesic agent to yield a variety of analgesic bisphosphonates.

The above suggested reactions are illustrative of several synthetic routes to achieve the chemical combination of the analgesic agent with the bisphosphonate.

Clinically, bisphosphonates act almost exclusively on bone because of their high affinity for calcium phosphate. Consequently, they are rapidly drawn to bone (bone seekers) and are strongly bound in the resorption lacunae. The major physicochemical effects of the bisphosphonates are decreased solubility of bone substance and changes in mineralization because of their incorporation into hydroxy apatite crystals and into bone matrix. Due to their affinity for and adherence to solid-phase calcium phosphate, bisphosphonates inhibit the formation, aggregations and dissolution of crystals. Clinically, but more important, therapeutic action of bisphosphonates is inhibition of bone resorption which commences within one to two days after administration regardless of the route and frequency of administration (oral or intravenously, daily, weekly, monthly, semi-annually). The total amount given determines the overall effect. The reduction in bone resorption is accompanied by a positive calcium balance, which is the justification for the use of these drugs in osteoporosis.

The mechanism of action of bisphosphonates are complicated and operate at the cellular and molecular levels.

Bisphosphonates inhibit local bone destruction by tumors and decrease tumor burden in bone. Previously it was thought that bisphosphonates had no direct effect on tumor cells. Recent studies reveal that they inhibit the adhesion and spreading of tumor cells in vitro. By combining the bisphosphonates with the analgesic agent we increase lipophilicity of the bisphosphonate, thereby increasing the bioavailability of the bisphosphonate which heretofore only 1-10 percent was available to the absorption of the body.

Bisphosphonates, when chemically combined with the analgesic, diminished bone pain, leading to a marked improvement in the quality of life, especially with those suffering from arthritic pain and the pain of cancer. The salicylates are widely used in the treatment of rheumatic and arthritic disorders. Analgesic abuse is often noted in patient with chronic gastrointestinal and renal disease. Many such patients are in the habit of taking analgesics for prolonged periods and usually in excessive doses. The bisphosphonates used in this invention are useful in the treatment of aforesaid pain and are potentiated by being chemically combined in the agent, which carries the analgesic directly to the site of the pain. Thus, the invention encompasses a means whereby a patient afflicted with tissue inflammation can secure relief without risking analgesic abuse due to over use of salicylates. The invention provides effective drug compositions and therapy and is based on the use of pharmacologically-active bisphosphonates reacted with salicylate based anti-inflammatory agents such as aspirin and ibuprofen, or narcotics. Within the scope of sound medical judgment, the dosage of analgesic bisphosphonate will vary with the particular condition being treated, the severity of the condition, the duration of the treatment, and the specific analgesic disphosphonate compound employed. While any pharmaceutically-acceptable salt of the bisphosphonates can be used in the practice of this invention, the sodium salts are preferred. Various pharmaceutical cations such as potassium, ammonium, mono, di, and triethanolammonium, and mixtures thereof are also suitable for use as counter ions in the salts. As can be seen from the forgoing, the preparation of the analgesic bisphosphonates used in the practice of this invention can be accomplished using well known methods or by simple modifications of various art-disclosed procedures. The compounds of the invention can be administered by all the known means, e.g., oral, intravenous, transdermal, intramuscular, suppository, or any medically acceptable means.

The reaction of chemically combining the agent can be done in many different ways, if the bisphosphomate is etidromate, alendronate, paridronate, ibandronate, zolendronate, there is a hydroxy group on that methylene bridge connecting the phosphonic groups. This OH group is reacted with aspirin acid chloride (acetylsalicyloyl chloride) or the acid chloride of ibuprofen (2-4-isobutyl phenyl)propionic acid chloride.

The reaction can also be achieved by using the carboxylic acid form of the analgesic compound as aspirin itself or ibuprofen itself, a catalyst as phosphoric acid or p-toluene sulfonic acid can be used an water removed and collected in a Dean Stark Trap.

If there is a chlorine, bromine or halogen on the methylene bridge, this can be reacted with the analgesic free acid, or the sodium, potassium or alkali or alkaline earth salt of the free acid. The same compounds can be prepared also by reacting the free acid, such as aspirin or ibuprofen with the hydroxy group with or without a catalyst as 85% phosphoric acid or by para toluene sulfonic acid. In some cases the bisphosphonic acid acts as its own catalyst. The reaction is facilitated by using an azeotroping agent to remove the water formed in the condensation reaction. The methyl or other esters of the analgesic agent also affords an alternate synthetic route. In this case methyl or other alcohol is removed at elevated temperature and a tin catalyst such as Fascat 4201 can be used.

For bisphosphonates not having the hydroxyl group, the products of the invention can be made by forming the metal derivatives using sodium metal, potassium metal or sodium hydride to react with the hydrogens on the methylene or alkylidine bridge connecting the two phosphorus atoms, and the then reacting that product with the analgesic acid or the acid chloride of the analgesic acid. These are acyloxy derivatives of bisphosphonates.

A still further means of combining the analgesic agent with the bisphosphonate is to form a quaternary compound from the following examples: ibandronate, minodronate, oltadronate, risondronate, zolendronate as bisphosphonates and positively charged amine analgesics.

Another means of combining the analgesic agent with the bisphosphonates is to react the analgesic agent or any suitable derivative with the amino group of those bisphosphonates, which contain an amino group such as alendronate, icandronate, neridronate, pamidronate. Preferably, the acid chloride of the analgesic acid is reacted with the amino group in a suitable solvent such as acetone, dimethyl foramide, dimethyl sulfoxide and other polar solvents. It is useful to use an acid acceptor such as pyridine or N,N, dimethyl amine, aq NaOH. The hydrochloride precipitates out and it filtered off. The product is isolated by evaporation of the solvent under vacuum under low temperatures.

Another way in which the analgesic agent can be attached to the bisphosphonate is to react a suitable derivative with one or more of the bisphosphonic groups by any means known to one familiar in chemistry. In all cases where the bisphosphonic acid term is used, it is meant to include salts and esters and chelates and other derivatives. The essential element of the invention is that we have two bisphosphonic acid groups attached to the same carbon atom.

An example of attaching an alkaloid type narcotic to a bisphosphonate is the formation of a mono, di, tri, or teltra ammonium salt. The formation of these organic salts also have a synergistic effect by enhancing the hydrophobicity of the particular derivatized bisphosphonate, thereby enhancing its bioavailability. Many amine containing gain reducing alkaloids are known and reported in the medical literature. Two useful specific examples by way of illustration are codeine and phenazocine.

Employing the same synthetic procedures, it is possible and desirable to react certain bisphosphonates having both a hydroxy and primary or secondary amino group forming the pain reducing conjugates of the bisphosphonates of this invention.

Still another aspect of the invention is to use the bisphosphonate compound as a moiety to carry therapeutic drugs to the bone area where they are carried by virtue of the bone seeking properties of the bisphosphonates. It is notable that these properties do not exist if the carbon (methylene) bridge between two phosphorus atoms is longer then one. For example, the ethylene 1,2 bisphosphonates are ineffective.

Any chemical connection of an analgesic agent to the bisphosphonates group is meant to be covered.

### EXPERIMENTAL

Structure I is a generalized formula encompassing all bisphosphonates, which are functional in preparing conjugates of this invention having anti-imflammory and/or narcotic properties. 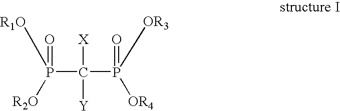
Where R₁, R₂, R₃ and R₄ independently are a straight or branched, optionally unsaturated C₁-C₇ alkyl, or C₂-C₇ alkenyl, hydrogen, or a pharmacologically acceptable salt, X=hydrogen, hydroxyl or halide, Y=hydrogen, halide or R-T (CH₂)n- where R is phenyl, pyridinyl, piperidinyl or pyrimidinyl which is unsubstituted or substituted by C₁-C₄ alkyl, C₁-C₄ alkoxy, halogen, hydroalkyl or nitro, T is S or primary, secondary or tertiary nitrogen NH and n=O, or T is a direct bond and n=1 to 6 provided that as a ring atom of the ring R and/or a chain atom of the group T, there is always at least one heteroatom from the group of NH and S, including the stereoisomers thereof.

The derivatives of this invention is based on the sound principle of covalently or ionically attaching a known analgesic compound to a variety of known bisphosphonates by well known organic chemical transformation. Anyone skilled in organic synthesis can easily carry out these conversions. There are several well-known types of organic reactions, which give the desired compositions of this invention. They involve the following:

Bisphosphonates containing a hydroxy group can be reacted with analgesic molecules that have an acid chloride, or acid functionality yielding esters.

Bisphosphonates with an amino group can also react with an acid chloride, of an analagesic compound but this time an amide is formed.

Mercaptide bisphosphonates will react with the same acid chlorides to yield thioesters.

Other transformations are also feasible, however the above reactions represent the easiest and simplest thereby serving as examples for the purpose of this invention.

The following synthetic discussion illustrates the teachings of this invention:

Bisphosphonates with a halide attached to the methylene carbon can react with a carboxylic acid or the corresponding carboxylic salt to give an ester derivative.

Methylene bisphosphonates can react with a strong base to form a fairly stable carbanion, which can subsequently react with a carboxylic acid or the corresponding acid chloride to also yield a conjugate ester containing a covalently attached analgesic moiety.

Still another simple chemical conversion to attach analgesic substances to bisphosphonates is to react nitrogenous painkillers with the osteoporosis bisphosphonate to form the corresponding ammonium salt. This can be a mono, di, tri or tetra salt depending on the reaction equivalents of the reagents.

There are many amine containing analgesic compositions. Specific examples include alfentanil, amidopyrine, codeine, levallorphan, lorfan and Talivin to mention such a few. There are many more examples of amine analgesics, which can be utilized by the teachings of this invention. Anyone experienced in organic and/or medicinal chemistry would be capable of deciding which can be utilized.

I claim:

1. Bisphosphonates compositions as shown in formula I 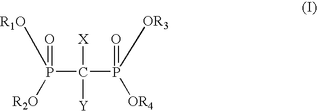
where R₁, R₂, R₃ and R₄ independently are a straight or branched, optionally unsaturated C₁-C₇ alkyl, or C₂-C₇ alkenyl, hydrogen, or a pharmacologically acceptable salt, X=hydrogen, hydroxyl or halide and Y=hydrogen, halide or R-T (CH₂)n- where R is phenyl, pyridinyl, piperidinyl, imidazinyl or pyrrolidinyl which is unsubstitated or substituted by C₁-C₄ alkyl, C₁-C₄ alkoxy, halogen, hydroalkyl or nitro, T is sulfur or nitrogen, and n=O, or T is a direct bond and n=1 to 6 provided that as a ring atom of the ring Y and/or a chain atom of the group T, there is always at least one heteroatom from the group of nitrogen or sulfur, including the stereoisomers, or a pharmacologically acceptable salt thereof, react with analgesic acid chlorides, carboxylic acids, or it's sodium salt, or ester, or analgesic amine containing molecules capable of forming ammonium salts thereof resulting in conjugates having medicinal properties of both pharmaceutical actives.

2. The conjugate drug of claim 1, wherein the bioactive bisphosphonate is selected from the group consisting of pamidronate, ibandronic, risedronate, cimadronate, clodronate, meridronate, olpadronate, pridronate, teludronate, zoleudronate, icadronate, alendronate or stidronate.

3. The conjugate drug of claim 1, wherein the bioactive analgesic compound is selected from the group consisting of acetylsalicylic acid, amidopyrine, antipyrine, condeine, propoxphene, hydromorphone, isobutylphenyl propionic acid, levallorphan, mefenamic acid, pentazocine, phenazopyridine, propiram, propoxyphene, sodium salicylate, alfentainl, bamadizon, clonixin, medetomidine, sodium diclofenac, morphine, epirizole, glofenine, levorphanol, medetomidine, niflumic acid, phenoperidine, sufentanil, tolmetic, naproxen, xylazine and the like.

4. A method to prepare the conjugates of claim 1, whereby a hydroxyl group of the bisphosphonate can be reacted with an acid chloride, carboxylic acid or ester containing analgesic moiety.

5. A method to prepare the conjugates of claim 1, whereby a primary or secondary amine of the bisphosphonate can be reacted with an acid chloride, carboxylic acid or ester containing analgesic moiety.

6. A method to prepare the conjugates of claim 1, whereby a hydroxy group and a primary or secondary amine, if present can be reacted with an acid chloride, carboxytic acid or ester containing analgesic moiety.

7. A method to prepare the conjugates of claim 1, whereby the methylene position of a bisphosphonate can be reacted with a strong base to form a carbanion followed by the reaction of an carboxylate anion of an analgesic moiety.

8. A method to prepare the conjugates of claim 1, whereby a halogen on the methylene position of a bisphosphonate can be reacted with a carboxylate anion of an analgesic moiety.

9. The use of compositions as described in claim 1 as inhibitors of bone resorption.

10. The use of compositions as described in claim 1 as inhibitors of tumor cells.

11. The use of compositions as described in claim 1 as inhibitors of inflammation and pain like in rheumatic and arthritic disorders.

12. A method to prepare the conjugates of claim 1, whereby a organic amine alkaloid or synthetic narcotic neutralizes one, two, three or four protons of the bisphosphonate.
